(19) United States
(12) Patent Application Publication
(10) Pub. No.: US 2006/0265029 A1
(43) Pub. Date: Nov. 23, 2006

(54) Apparatus and methods for using self-heating chamber to excite a multi-compound TDP layer to generate infrared wavelength for various pain treatment, and composition of this TDP layer

(75) Inventors: Lan Huang, Bronx, NY (US); Jilun Wu, Chong Qing (CN)

Correspondence Address: LAN HUANG, Suite 8J, 2500 Johnson Ave., Bronx, NY, 10463, US

(73) Assignee: TCM Groups Inc.

(21) Appl. No.: 11/134,976

(22) Filed: May 23, 2005

(51) Int. Cl. A61N 5/06; A61F 7/00; A61F 7/12

(52) U.S. Cl. 607/88; 607/96

(57) ABSTRACT

An apparatus, product formulation, and method for using heat to excite a multi-compound layer to generate infrared wavelength, wherein the apparatus includes 1) a multi-compound TDP layer which can be excited by heat to generate infrared wavelength and 2) a heat-generating chamber separated by a first non-permeable wall, wherein the reservoir and chamber are formed in or supported by a housing. The TDP layer is composed of more than 30 compounds including ZrO, SiO2, SiC2, Al2O3, FeO, MnO2, Cu2O, MgO, ZnO, and Cr2O3. The heat-generating chamber includes a medium for generating controlled heat, preferably a chemical composition made of carbon, iron, water, and/or salt, which is activated upon contact with air (oxygen). The heat can last as long as 16 hours. The function of the heat-generating element is to supply energy to excite TDP multi-compound layer to generate infrared wavelength at 2-18 micrometer (especially at 4-14 micrometer) range, for all types of pain treatment. The apparatus may also include a layer contacting user's skin to store active pharmaceuticals and transdermal system to additionally relieve pain and improve symptoms associated with arthritis, soft tissue damage, neck vertebrate pain, lumber muscle strain, lumber intervertebrate disc herniation, arthritis around shoulder, prostate inflammation, pelvic inflammation, pain of bone hyperplasia, gastroenteritis, and dysmenorrhoea.

### PREVIOUS PROVISIONAL PATENT

Filed on May 29, 2004, application No. 60/576,075

### REFERENCES

Improvement of pain and disability in elderly patients with degenerative osteoarthritis of the knee treated with narrow-band light therapy, Stelian J et al., J Am Geriatr Soc. 1992 January;40(1):23-6.

Infrared laser in the treatment of craniomandibular disorders, arthrogenous pain. Hansson T L. J Prosthet Dent. 1989 May;61(5):614-7.

Effect of different methods of thermotherapy on skin microcirculation. Berliner M N, Maurer A I. Am J Phys Med Rehabil. 2004 April;83(4):292-7.

Rheumatoid arthritis-affected temporomandibular joint pain analgesia by linear polarized near infrared irradiation, Yokoyama K, Oku T. Can J Anaesth. 1999 July;46(7):683-7.

### BACKGROUND OF THE INVENTION

1. Field of the Invention

The present invention relates to novel composition of multi-compound TDP layer, which can be excited by heat to generate strong enough infrared wave which can relieve pain. The invention also relates to novel portable devices and methods to relieve pain using self-heating chamber at a certain temperature and infrared wave at certain wavelength and certain intensity for an extended time.

2. State of the Art

It is known that elevated temperature can relieve pain, which is an approved method by FDA for pain relief. It is also shown in publications that infrared wave (or light) can relieve pain.

Infrared light increases energy inside cells by stimulating cytochromes, which are part of the electron transportation chain that converts sugar into energy. This stimulates cellular reproduction, relaxes muscles and stimulates nerve transmission, enhances the immune response, reduces scar tissue and promotes wound healing, stimulates collagen production (making skin tighter), increases blood capillary circulation and vascular activity, stimulates production of endorphins and enkephelins from the brain, increases RNA and DNA synthesis, stimulates production of the adrenals which reduces pain and stimulates the production of adenosine triphosphate (ATP), an immediate energy source for muscle contraction.

Additionally there have not been any reported negative side effects. Infrared treatment is being used for arthritis, wounds, and burns, against cancer, for bone loss, diabetic ulcers, poor blood or oxygen supply. The treatment is very effective for pain and inflammation reduction. There are six studies of subjects who had lost their protective sensation in their lower limbs due to diabetic neuropathy. All the limbs actively treated with infrared showed improvement. The placebos in the double-blind studies showed almost no improvement. (http://www.garyascott.com/archives/2003/02/18/765)

So far all infrared wave treatment devices are bulky and heavy, usually requiring medical doctor's assistance in using them. Some innovation came recently in reducing the size of infrared light treatment devices, but they still are hand-held, bulky, and need to be powered by electricity or batteries. According to a recent patent (No. 6,162,215), an infrared rays from a hand-held cauterizer device can be located in proximity of a specific point of the human body without attendance of a medical personnel. The base is provided in the interior thereof with a plurality of temperature controllers and timers for regulating the treatment temperature and the treatment duration of a heater of the cauterizers. The heater is capable of bringing about a predetermined energy for treating disease or relieving pain of the specific point of the body of a patient under treatment. Another recent patent (No. 6,019,482) displayed a hand-held, self-contained irradiator powered by batteries. At an applicator end are provided many diodes that emit electromagnetic radiation in the visible and/or infrared portions of the spectrum. A series of switches are provided so that the user may select which one or ones of the diodes to activate to provide particular wavelengths or colors of radiation to be emitted from the applicator end to be used to treat particular body surface areas for the relief of pain or other problems.

Literatures have shown the effectiveness of infrared light in relieving pain. In a study conducted by J. Stelian et al. from Geriatric Medical Center, Shmuel Harofe Hospital, Beer Yaakov, Israel, researchers observed improvement of pain and disability in elderly patients with degenerative osteoarthritis of the knee treated with narrow-band light therapy (J. Am Geriatr Soc. 1992 January;40(1):23-6.). DESIGN: Partially double-blinded, fully randomized trial comparing red, infrared, and placebo light emitters. PATIENTS: Fifty patients with degenerative osteoarthritis of both knees were randomly assigned to three treatment groups: red (15 patients), infrared (18 patients), and placebo (17 patients). Infrared and placebo emitters were double-blinded. INTERVENTIONS: Self-applied treatment to both sides of the knee for 15 minutes twice a day for 10 days. MAIN OUTCOME MEASURES: Short-Form McGill Pain Questionnaire, Present Pain Intensity, and Visual Analogue Scale for pain and Disability Index Questionnaire for disability were used. Researchers evaluated pain and disability before and on the tenth day of therapy. The period from the end of the treatment until the patient's request to be retreated was summed up 1 year after the trial. RESULTS: Pain and disability before treatment did not show statistically significant differences between the three groups. Pain reduction in the red and infrared groups after the treatment was more than 50% in all scoring methods (P<0.05). There was no significant pain improvement in the placebo group. Researchers observed significant functional improvement in red- and infrared-treated groups (P<0.05), but not in the placebo group. The period from the end of treatment until the patients required treatment was longer for red and infrared groups than for the placebo group (4.2+/−3.0, 6.1+/−3.2, and 0.53+/−0.62 months, for red, infrared, and placebo, respectively), with infrared group being the longest. CONCLUSIONS: Low-power light therapy is effective in relieving pain and disability in degenerative osteoarthritis of the knee, with infrared light therapy being the most effective.

In another study conducted by T L Hansson at University of Amsterdam, Academic Centre for Dentistry, The Netherlands showed that infrared laser in the treatment of craniomandibular disorders, arthrogenous pain. (J Prosthet Dent. 1989 May;61(5):614-7). The fast removal of intra-articular inflammation of the temporomandibular joint in five different patients after infrared laser application is observed. One parameter of clinical evaluation was maximum mouth opening and subjective pain. The application of infrared laser of 700 Hz frequency for 3 minutes during five consecutive days at the skin over the painful area of the temporomandibular joint was used. However, the importance of concomitant mandibular stabilization is stressed to achieve optimal result.

In the following study conducted by K. Yokoyama et al. at Department of Anesthesia, School of Dentistry, Kagoshima University Dental Hospital, Sakuragaoka, Japan (Can J Anaesth. 1999 July; 46(7):683-7), Rheumatoid arthritis-affected temporomandibular joint pain analgesia by linear polarized near infrared irradiation was evaluated. CLINICAL FEATURES: They investigated four female patients (age 42.8+/−26.0 yr) with chronic rheumatoid arthritis affecting a single TMJ. Patients had received antirheumatic drugs such as sodium aurothiomalate, and as a result showed no symptoms in other body joints. Linear polarized near infrared radiation using Super Lizer was applied weekly with and/or without jaw movement to the unilateral skin areas overlying the mandibular fossa, anterior articular tubercle, masseter muscle and posterior margin of the ramus of the mandible. The duration of irradiation to each point was two seconds on and ten seconds off per cycle and the intensity at each point was approximately 138 J×cm(−2) at a wavelength of 830 nm. Interincisal distance was measured with maximal mouth opening in the absence and presence of pain before and after each treatment. Additionally, subjective TMJ pain scores assessed using a visual analog scale were performed for painful maximal mouth opening before and after each irradiation. TMJ pain disappeared after only four treatments. Moreover, painless maximal mouth opening without pain after irradiation in three patients was on average improved to 5.3+/−2.1 mm. However, one case was observed where the opening length prior to irradiation did not improve, despite the fact that the RA-affected TMJ pain had disappeared. CONCLUSION: Application of linear polarized near infrared irradiation to patients with RA-affected The pain is an effective and non-invasive short-term treatment.

Skin microcirculation was investigated in this study conducted by MN Berliner et al at Third Department of Internal Medicine (MNB) and the Department of Orthopaedics and Orthopaedic Surgery (AIM), University Hospital Giessen, Giessen, Germany (Am J Phys Med Rehabil. 2004 April;83(4):292-7). OBJECTIVE: The purpose of the study was to compare the effects of different methods of thermotherapy on skin microcirculation, skin temperature, and core temperature in patients with rheumatoid arthritis and in healthy persons. DESIGN: In 20 patients with rheumatoid arthritis and 20 healthy controls, the skin microcirculation was measured by means of laser-Doppler flowmetry before, at the end of, and 30 mins after completion of local thermotherapy (infrared light, paraffin, peat) in the lumbar area. In addition, skin and core temperature were recorded at the same point in time. The influence of previous intake of acetylsalicylic acid on skin microcirculation during thermotherapy was investigated. The controlled trial was conducted in an air-conditioned laboratory. RESULTS: All methods caused a significant increase in skin microcirculation (flow and vasomotion), skin temperature, and core temperature. The skin temperature showed the highest increase when infrared light was applied. The microcirculation became more intense when paraffin fango was used. There were no differences between the reactions of rheumatoid arthritis patients and healthy persons. The additional intake of 500 mg acetylsalicylic acid before treatment did not affect skin temperature or skin microcirculation. A tendency toward a sinusoidal rhythmization of vasomotion was detected. CONCLUSIONS: The effects of the three methods of thermotherapy differ quantitatively due to their physical attributes. The effects on circulation differ depending on the method chosen, and this is significant when selecting the least stressful method for a particular patient. Rheumatoid arthritis patients and healthy controls have the same increase of skin microcirculation in the lumbar area. Findings in the distal limbs might differ. All used therapies cause a rhythmization of the vasomotion and, as a consequence, a qualitative improvement of the microcirculation.

A newly FDA approved battery-charged device, Wrap-10 from Quantum Devices Inc. (www.quantumdec.com) relieves pain with high and optimum intensity infrared wave (50 mw/cm²). However, it is very bulky, with outside dimensions at 5.55 in. (141 cm)×2.65 in. (67 mm)×2.0 in. (51 mm), and a weight of 1.2 pounds (549 g) with batteries.

Far IR (FIT) therapy was approved by FDA for pain treatment. DUOKE patch radiates IR in the Far IR range (2-18 micron range). At the molecular level, Far IR exerts strong rotational and vibrational effects that are biologically beneficial.

Although the wavelengths of FIR are too long for the eyes to perceive, we can experience its energy as gentle, radiant heat, which can penetrate up to [3.5] inches beneath the skin. Among FIR's healing benefits is its ability to stimulate inflammation, which is necessary for a period of time in order to heal injuries such as a pulled muscle. FIR also appears capable of enhancing white blood cell function, thereby increasing immune response and the elimination of foreign pathogens and cellular waste products. Additional benefits include the ability to stimulate the hypothalamus, which controls the production of neurochemicals involved in such biological processes as sleep, mood, pain sensations, and blood pressure; enhancing the delivery of oxygen and nutrients to the body's soft tissue areas; and the removing accumulated toxins by improving lymph circulation [Alternative Medicine, page 670].

It is well known that infrared wave treatment can relieve pain according to the above publications and approved patents. However, none of the prior art systems use self-heating chamber to excite certain compounds to generate infrared wave. And the current infrared physical treatment uses bulky machines powered by electricity that are not portable. Most of the large devices heat up to over 300 degree C., which are dangerous at close distance. Thus they have to be positioned at a distance to the treatment area. And the treatment time is limited to less than 2 hours because of in-house treatment. Some portable devices use large batteries (Wrap-10 example), thus lending heavy weight.

Therefore, it would be advantageous to develop an apparatus and method which achieves the same treatment effect using light, portable, easy to use patches with long treatment time. We have invented just that, a portable infrared wave treatment patch, which is light (25 g), small (6 cm×10 cm×0.5 cm), user friendly (just peel off the container, and let the patch contact air), long lasting (up to 16 hours of heat and IR treatment (IR intensity of 54 mw/cm² at 8 micrometer)), synergized with heat treatment (regulated warm temperature up to 16 hours, with self-heating chamber by contacting to air). This patch can further include active pharmaceutical layer with desired transdermal technology to deliver anti-pain drugs to the pain area.

### SUMMARY OF THE INVENTION

The present invention provides many important advantages over prior art. We invented a portable infrared light patch system to relieve pain using both heat and infrared wave. The infrared wave is generated by the flexible multi-compound TDP layer excited by heat. TDP layer includes, but not limited to ZrO, SiO2, SiC2, Al2O3, FeO, MnO2, Cu2O, MgO, ZnO, and Cr2O3, up to more than 30 compounds. The infrared wavelength generated is between 2-18 micrometer (the highest intensity at 4-14 micrometer).

The present invention provides novel apparatus and formulations, and the method of using same, for achieving improved therapeutic effect by using infrared wave combined with regulated heat. One aspect of the present invention is an infrared wave treatment device which has a multi-compound TDP layer (for generating infrared wave) and a heat-generating chamber separated by a non-permeable wall.

The heat-generating chamber (heating element) includes means for generating controlled heat (electrical, chemical, etc.). Preferably, the heat-generating means is a chemical composition made of carbon, iron, water and/or salt which is activated upon contact with air (oxygen). The preferred heat-generating chamber has means for allowing the heat-generating medium to have limited and controlled contact with ambient air, such as a cover or housing with opening(s), areas with semipermeable membrane(s) or entire surfaces made of semipermeable membrane(s). The advantages offered by this chemical type of heat-generating composition, when used in the apparatus of this application, include high thermal energy per unit mass, rapid onset of heating, stable and controllable heating temperature over extended duration, light weight, independent operation (i.e., no need to be hooked up to any machine such as is necessary in electrical heating systems), and relatively low cost.

To use this infrared wave and heat device, the user activates the heat-generating chamber (with the preferred embodiment, the user exposes the device to air by removing it from an air-tight storage container), after which the user affixes the device adjacent to the skin area. The device can also be made such that it has a drug reservoir, which is between the TDP layer and the self-heating layer.

The infrared wave and self-heating device of the invention affects heating the device and skin to a desired narrow temperature range for sufficient time to achieve rapid excitation of infrared wave from the multi-compound TDP layer. It is very convenient to use (i.e., light weight, small volume, not hooked up to other electrical equipment or battery, easy to dispose because of use of environmentally safe materials, etc.).

Our self-heating infrared wave pad is a physical therapy method, combining Chinese traditional moxibustion with electromagnetic infrared wave of TDP layer excited by self-heating energy.

All in all, we have invented a simple, portable, effective pain treatment patch combing controlled heat and effective IR wave, with the following advantages over prior products: 1) portable, 2) light (25 g), 3) small (6 cm×10 cm×0.5 cm), 4) user friendly, and 5) longest lasting (up to 16 hours of heat and IR treatment (IR intensity of 54 mw/cm² at 8 micrometer)).

The advantages are detailed as the following:
1) Our patch is effective and safe. The TDP layer is flexible and soft, which is suitable for treating uneven parts of human body. The temperature is stable at 38 to 60 degree C. (for extended time, up to 16 hours). The effective infrared wave (with wavelength over a wide 2-18 micrometer ranges, and intensity at 54 mw/cm² at 8 micrometer point) can invigorate and promote the circulation of blood, diminish inflammation, promote metabolism. It is ideal to treat pain in users' neck, shoulder, waist, stomach, and leg, with over 75% response rate.
2) Our patch is easy to use and is portable. It is quite inconvenient to go to hospital for physical therapy every day. Our patch can be used anywhere, because it is light (25 g) and its treatment effect lasts a long time (up to 16 hours).
3) Our patch is the first product using self-heating chamber to excite the TDP layer to generate infrared wave. In addition, heat treatment for pain is synergistic with the infrared wave treatment.
4) The TDP layer compound is stable at room temperature.
5) Additional active pharmaceutical layer can be added between the TDP layer and the self-heating chamber for additional pain relieving effect.

### BRIEF DESCRIPTION OF THE DRAWINGS

FIG. 1 Front and side view of our patch. Layers include 1) protective layer; 2) self-heating chamber; 3) insulation layer; 4) optional layer with active pharmaceuticals; 5) TDP layer; 6) adhesive patch; 7) holes for air.

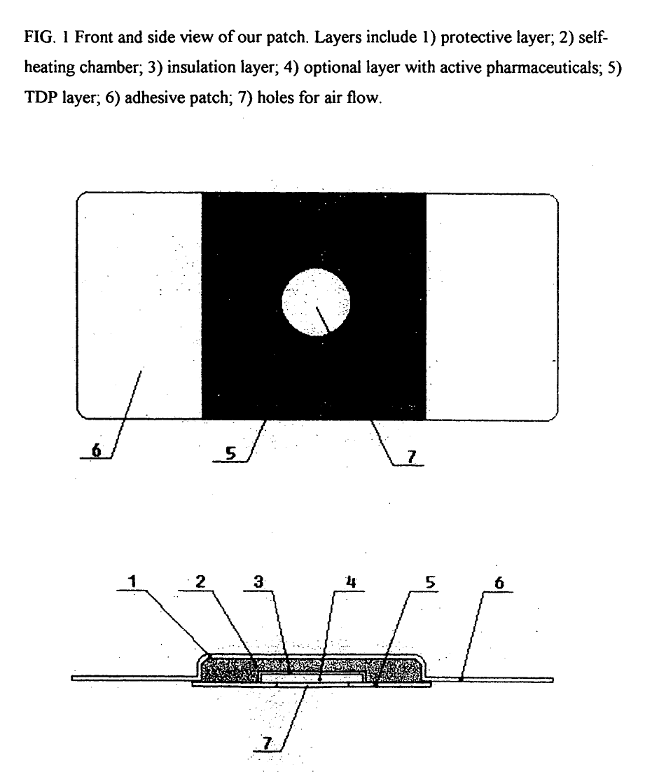

FIG. 2 Temperature curves of our patch “DUOKE” versus “Thermacare” patch.

FIG. 3 Infrared radiation intensity of our patch with TDP layer vs. without.

### EXAMPLES

### Example 1

Self-Heating Temperature and Time Duration

Chongqing Institute of Supervision & Inspection on Product Quality (QA) conducted examination on our patch products. From 200 pieces of patches manufactured on Mar. 2, 2004, Quality Assurance Department (QA) chose 12 pieces according to Chinese government medical device standard (YZB/Yu 0005-2002). The examination was conducted on Mar. 11, 2004. The results show that 1) the weight of TDP layer is greater than 30 mg (around 32 mg); 2) the highest temperature for the patch is less than 65 degree C. (around 61 degree C.); 3) the high temperature persists for over 16 hours; and 4) the average temperature on the patch surface is between 38 to 50 degree C.

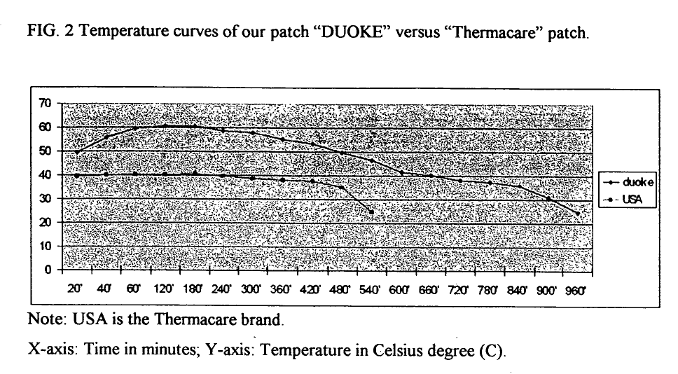

The temperature curve is shown in FIG. 2. Comparing with the top selling heat patch in the US “Thermacare”, “DUOKE” TDP self-heating patch self-heats at a higher temperature and lasts much longer. The self-heating process of “DUOKE” can last 16 hours compared with 8 hours in “Thermacare”. The temperature chart of both products is shown in FIG. 2.

### Example 2

Infrared Wave Intensity Detection

In two examinations reports (one on Nov. 8, 2001, the other on Oct. 31, 2003) from renowned Shanghai Physics Technology Institute of China Academy of Science, our patch products showed consistent radiation of infrared wave, with peak at 9-11 micrometers, detected with IRE-1 Infrared Radiation Detection Device.

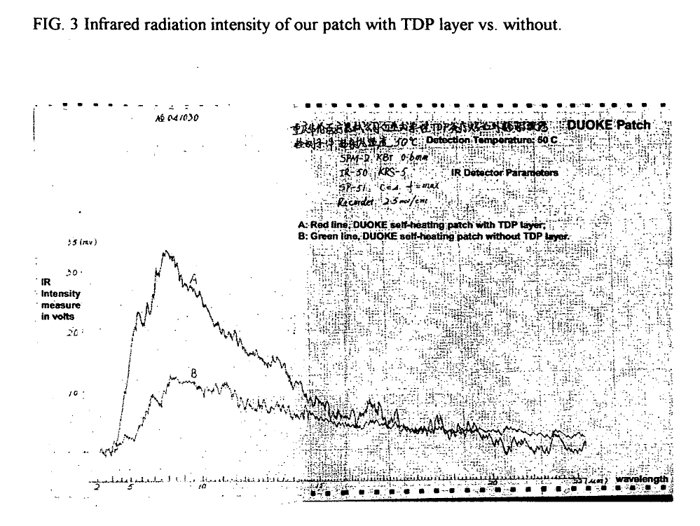

In a recent test conducted in Kunming Physics Research Institute, the infrared radiation intensity was compared in our self-heating patch with and without the TDP layer. According to the data shown in FIG. 3, at the peak of radiation intensity, the IR intensity of DUOKE patch with TDP layer (around wavelength 8 um) is 53.7 mw/cm², and that of DUOKE patch without TDP layer is 23.4 mw/cm².

IR radiation intensity of over 50 mw/cm² is high enough to relieve pain. One FDA approved portable IR pain-relieving, battery-charged product “Quantum Wrap-10” manufactured by Quantum Devices, Inc. has optimum IR intensity at 50 mw/cm2.

Comparing with Wrap-10, DUOKE TDP self-heating patch is superior because of its small size (6 cm×10 cm×0.5 cm), light weight (50 g), long lasting (16 hours), portability, and affordability.

### Example 3

Clinical Experimental Data.

Many clinical studies have been carried out to demonstrate the effectiveness and safety of our TDP infrared patch products. Below are two of them.
1) A study on 528 patients treated with our self-heating and flexible TDP adhesive patch was conducted in Academy of Traditional Chinese Medicine at Chongqing, China.

This hospital used our patch (trade name: DUOKE Self-heating Adhesive Pad) on their patients, from April 2002 to May 2003.

Number of patients enrolled: 528 (272 males and 256 females).

Average age: 42 years of age, ranging from 15 to 81.

Diseases treated: soft tissue damage (51 patients), neck vertebrate pain (53 patients), lumber muscle strain (55 patients), lumber intervertebrate disc herniation (54 patients), arthritis around shoulder (54 patients), prostate inflammation (55 patients), pelvic inflammation (51 patients), pain of bone hyperplasia (51 patients), rheumatic arthritis (52 patients), and gastroenteritis (52 patients).

Investigation methods: The treatment area was cleaned first with 75% alcohol. The patch was applied to the treatment area with the TDP layer close to the skin. Each patch was left on for one day. For acute pain, patients were treated for 1-3 days; while for chronic pain, patients were treated for 4-6 days. The adverse events were collected.

Criteria for efficacy: a) Complete response (CR): clinical symptoms and pain completely disappeared; b) Partial response (PR): clinical symptom improved and pain diminished noticeably; c) Minor response (MR): clinical symptom improved a little and pain diminished somewhat; d) No response (NR): no change in clinical symptom and pain, or worsened.

TABLE 1
Treatment Response of Our Invention:
DUOKE Self-heating Adhesive Pad

| | | | | | | Response |
|---|---|---|---|---|---|---|
| Disease | Patients | CR | PR | MR | NR | Rate |
| Soft tissue | 51 | 19 | 23 | 5 | 4 | 92.2% |
| damage | | | | | | |
| Neck | 53 | 8 | 13 | 20 | 12 | 77.4% |
| vertebrate | | | | | | |
| pain | | | | | | |
| Lumber | 55 | 18 | 15 | 15 | 7 | 87.3% |
| muscle strain | | | | | | |
| Lumber | 54 | 3 | 8 | 25 | 18 | 66.7% |
| intervertebrate | | | | | | |
| disc | | | | | | |
| herniation | | | | | | |
| Arthritis | 54 | 12 | 11 | 21 | 10 | 81.5% |
| around | | | | | | |
| shoulder | | | | | | |
| Prostate | 55 | 9 | 12 | 22 | 12 | 78.2% |
| inflammation | | | | | | |
| Pelvic | 51 | 6 | 11 | 15 | 19 | 62.8% |
| inflammation | | | | | | |
| Pain of bone | 51 | 9 | 17 | 20 | 5 | 90.2% |
| hyperplasia | | | | | | |
| Rheumatic | 52 | 10 | 19 | 14 | 9 | 82.7% |
| arthritis | | | | | | |
| Gastroenteritis | 52 | 8 | 18 | 16 | 10 | 80.8% |
| Total | 528 | 102 | 147 | 173 | 106 | 79.9% |

Therefore, the overall CR, PR, and response rates are 19.32% and 27.84%, and 79.9%, respectively. Our patch product is effective.

In addition, no adverse effects were reported.
2) A study on 515 patients treated with our self-heating and flexible TDP adhesive pad, was conducted in Herbal and Western Medicine Hospital in Chongqing.

This hospital used our patch (trade name: DUOKE Self-heating Adhesive Pad) on their patients, from April 2002 to May 2003.

Number of enrolled patients: 515 (261 males and 254 females).

Age: between 13 to 83 years of age.

Diseases treated: soft tissue damage (52 patients), neck vertebrate pain (51 patients), lumber muscle strain (54 patients), lumber intervertebrate disc herniation (51 patients), arthritis around shoulder (50 patients), prostate inflammation (53 patients), pelvic inflammation (51 patients), pain of bone hyperplasia (51 patients), rheumatic arthritis (52 patients), and gastroenteritis (51 patients).

Investigation methods: The treatment area was cleaned first with 75% alcohol. The patch was applied to the treatment area with the TDP layer close to the skin. Each patch was left on for one day. For acute pain, patients were treated for 1-3 days; while for chronic pain, patients were treated for 4-6 days. The adverse events were collected.

Criteria for efficacy: a) Complete response (CR): clinical symptoms and pain completely disappeared; b) Partial response (PR): clinical symptom improved and pain diminished noticeably; c) Minor response (MR): clinical symptom improved a little and pain diminished somewhat; d) No response (NR): no change in clinical symptom and pain, or worsened.

TABLE 2
Treatment Response of Our Invention:
DUOKE Self-heating Adhesive Pad

| | | | | | | Response |
|---|---|---|---|---|---|---|
| Disease | Patients | CR | PR | MR | NR | Rate |
| Soft tissue | 52 | 21 | 18 | 8 | 5 | 90.4% |
| damage | | | | | | |
| Neck | 51 | 10 | 9 | 20 | 12 | 76.5% |
| vertebrate | | | | | | |
| pain | | | | | | |
| Lumber | 54 | 17 | 16 | 12 | 9 | 83.3% |
| muscle strain | | | | | | |
| Lumber | 51 | 2 | 15 | 16 | 18 | 64.7% |
| intervertebrate | | | | | | |
| disc | | | | | | |
| herniation | | | | | | |
| Arthritis | 50 | 12 | 13 | 17 | 8 | 84.0% |
| around | | | | | | |
| shoulder | | | | | | |
| Prostate | 53 | 9 | 13 | 20 | 11 | 79.2% |
| inflammation | | | | | | |
| Pelvic | 51 | 5 | 13 | 15 | 18 | 64.7% |
| inflammation | | | | | | |
| Pain of bone | 50 | 9 | 13 | 18 | 10 | 80.0% |
| hyperplasia | | | | | | |
| Rheumatic | 52 | 8 | 19 | 15 | 10 | 80.8% |
| arthritis | | | | | | |
| Gastroenteritis | 51 | 8 | 10 | 18 | 15 | 80.8% |
| Total | 515 | 101 | 139 | 159 | 116 | 77.5% |

Therefore, the overall CR, PR, and response rates are 19.6% 27.0%, and 77.5%, respectively. Our patch product is effective.

In addition, no adverse effects were reported.

What is claimed is:

1. A portable, non-battery powered apparatus for generating infrared wavelength comprising:
a) a hollow frame having a first open end and a second open end;
b) a non-permeable wall disposed between said hollow frame's first and second open ends;
c) a first chamber defined by said hollow frame, said non-permeable wall, and said hollow frame first open end;
d) a second chamber defined by said hollow frame, said non-permeable wall, and said hollow frame second open end, said second chamber configured for housing a multi-compound TDP layer which can be excited by heat to generate infrared wavelength; and
e) a heating element for generating energy to excite the multi-compound TDP layer, thereof, said heating element comprising a heat-generating means disposed within said first chamber.

2. The apparatus of claim 1, wherein said heat-generating means is a heat-generating medium comprising a chemical composition made of carbon, iron, water, salt, or a combination thereof which is activated upon contact with ambient air.

3. The apparatus of claim 2, wherein said hollow frame first open end is capped with a structure for controlling ambient air contact with said heat-generating medium.

4. The apparatus of claim 3, wherein said structure comprises a cover non-permeable to air with opening(s) therein, or area(s) comprising a membrane with desired permeability to air.

5. The structure in claim 4, wherein said cover is made of material(s) having thermal insulation properties.

6. The apparatus of claim 4, wherein the structure further comprises means for adjusting the effective surface area of the opening(s) or portion(s) comprising a membrane with desired permeability to air.

7. The apparatus of claim 1, wherein said heating element is capable of exciting the multi-compound TDP layer to generate infrared wavelength, including but not limited to wavelength at 2-18 micrometer.

8. The apparatus of claim 1, wherein said heating element is adapted to maintain said temperature (38-50 degree C., and <65 degree C.) within a desired temperature range for a predetermined length of time.

9. The apparatus of claim 1, where said multi-compound TDP layer includes, but not limited to, ZrO, SiO2, SiC2, Al₂O3, FeO, MnO2, Cu2O, MgO, ZnO, and Cr2O3, up to more than 30 compounds.

10. The apparatus of claim 1, wherein said hollow frame further includes an adhesive surface adjacent to said hollow frame second open end.

11. The apparatus of claim 1, further including a pharmaceutically-active formulation, including herbal medicine, or OTC drugs, or patented drugs with any transdermal technology in a layer between the TDP layer and the self-heating chamber.

12. A method for generating infrared wavelength using self-heating chamber in a portable patch comprising:
a) a hollow frame having a first open end and a second open end;
b) a non-permeable wall disposed between said hollow frame's first and second open ends;
c) a first chamber defined by said hollow frame, said non-permeable wall, and said hollow frame first open end;
d) a second chamber defined by said hollow frame, said non-permeable wall, and said hollow frame second open end said second chamber configured for housing multi-compound TDP layer which can be excited to generate infrared wavelength; and
e) a medium for heating and regulating the heating temperature, said heat-generating medium disposed within said first chamber;
activating said heating medium;
affixing said patch to said user's skin such that said multi-compound TDP layer is in direct contact therewith the user's skin; and
allowing said patch to remain on said user's skin for a predetermined duration of time.

13. The method of claim 12, wherein said heat-generating medium is a chemical composition made of carbon, iron, water, salt, or a combination thereof which is activated upon contact with ambient air.

14. The method of claim 12, further comprising heating the patch and regulating the temperature thereof into a desired and elevated narrow range for extended length of time.

15. The method of claim 14 further comprising controlling the rate of heat-generation of said heat-generating medium subsequent to application to said user's skin.

16. The method of claim 12 further comprising adhering said patch to said user's skin.

17. The method of using heat at regulated temperature and infrared wavelength together in a portable format for an extended time to treat pain, including but not limited to, pain at neck, shoulder, knee, stomach, waist, or arthritis. The method can additionally relieve symptoms in soft tissue damage, neck vertebrate pain, lumber muscle strain, lumber intervertebrate disc herniation, arthritis around shoulder, prostate inflammation, pelvic inflammation, pain of bone hyperplasia, gastroenteritis, and dysmenorrhoea.

18. The apparatus of claim 14, wherein said heat-generating medium is an exothermic chemical medium activated by contact with oxygen in the ambient air.
